(19) United States
(12) Patent Application Publication
(10) Pub. No.: US 2004/0138680 A1
(43) Pub. Date: Jul. 15, 2004

(54) Microdermabrasion apparatus and system

(76) Inventors: David J. Twitchell, Snellville, GA (US); Richard H. Ford, Braselton, GA (US); Eric D. Fuller, Smyrna, GA (US)

Correspondence Address: HINKLE & O'BRADOVICH, LLC, 395 SCENIC HIGHWAY, LAWERENCEVILLE, GA, 30045, US

(21) Appl. No.: 10/722,330

(22) Filed: Nov. 25, 2003

Related U.S. Application Data
(60) Provisional application No. 60/429,773, filed on Nov. 26, 2002.

(51) Int. Cl. A61B 17/50

(52) U.S. Cl. 606/131

(57) ABSTRACT

A microdermabrasion apparatus and system is provided. In general, the apparatus includes a housing having a first end and a second end, a suction cup located at the first end of the housing, a motor located within the housing, a pump located within the housing, an exfoliation tip mechanically coupled to a shaft that is mechanically coupled to the motor, the exfoliation tip being located within a vacuum space in the suction cup and a tube located within the housing and connected between the pump and the suction cup.

Priority is claimed based on U.S. Provisional Patent Application, Serial No. 60/429,773, filed Nov. 26, 2002, and entitled “Microdermabrasion Apparatus and System”.

### BACKGROUND

I. Field of the Invention

The present invention relates generally to the field medical microdermabrasion and more particularly to a personal use microdermabrasion apparatus and system.

II. Description of the Related Art

Microdermabrasion systems are available for exfoliating skin thereby providing advantages such as increased blood flow and the removal of dead skin cells. There are several disadvantages with modem microdermabrasion systems. Many commercially available microdermabrasion systems can be very costly. Furthermore, many of the present systems require that the skin be hydrated during use. Many systems also require that internal motors and vacuum pumps be coupled to one another to ensure operation of the system. Yet another disadvantage is that many systems require that a disposable filter be used and changed on a periodic basis.

### SUMMARY

In general, the invention features a personal use microdermabrasion apparatus and system that provides a user with a small, convenient and inexpensive skin exfoliator with attachments.

In general, in one aspect, the invention features a microdermabrasion apparatus, including a housing having a first end and a second end, a suction cup located at the first end of the housing, a motor located within the housing, a pump located within the housing, an exfoliation tip mechanically coupled to a shaft that is mechanically coupled to the motor, the exfoliation tip being located within a vacuum space in the suction cup and a tube located within the housing and connected between the pump and the vacuum space.

In one implementation, the apparatus further includes a power entry optionally connected to the second end of the housing, the power entry being electrically coupled to a switch located on the housing, to the pump and to the motor.

In another implementation, the apparatus further includes a cylindrical wall located adjacent the first end and within a portion of the suction cup.

In another implementation, the apparatus further includes a seal located around the perimeter of the wall and in between the wall and a portion of the suction cup.

In another implementation, the seal is an o-ring.

In another implementation, the tube protrudes from a portion of the wall into the vacuum space.

In still another implementation, the apparatus further includes a filter located within the tube.

In yet another implementation, the apparatus further includes a vent located in the tube between the filter and the vacuum space.

In another implementation, the exfoliation tip comprises a main body connected to the shaft and an abrading impregnated surface disk connected generally perpendicular to the main body.

In another implementation, the abrading impregnated surface disk further comprises an abrasive media.

In another implementation, the suction cup further comprises an opening generally defined by a ridge.

In another implementation, the exfoliation tip is offset inside the vacuum space and from the ridge by a distance.

In another aspect, the invention features a microdermabrasion kit, including a microdermabrasion apparatus having a housing having a first end and a second end, a cylindrical wall located generally adjacent the first end, a motor located within the housing, the motor including a shaft, a pump located within the housing and a tube located within the housing and connected between the pump and the cylindrical wall, a suction cup adapted to be connected to the wall and an exfoliation tip adapted to be connected to the motor shaft.

In one implementation, the exfoliation tip is adapted to be located within a vacuum space formed when the suction cup is placed over the wall and the pump pulls a vacuum on the space, the exfoliation tip being further adapted to spin when the motor is supplied power.

In another implementation, the exfoliation tip comprises an abrading impregnated surface disk.

In another implementation, the kit further includes a pore cleansing tip adapted to be connected to the shaft.

In another implementation, the kit further includes a buffer tip for finger nails and toe nails adapted to be connected to the shaft.

In another implementation, the kit further includes a sander tip for finger nails and toe nails adapted to be connected to the shaft.

In another implementation, the kit further includes a pumice stone adapted to be connected to the shaft.

In yet another implementation, the kit further includes a vibrational tip adapted to be connected to the shaft.

In still another implementation, the suction cup is abrasive.

In another implementation, the suction cup includes an opening generally defined by a ridge.

In another implementation, the ridge includes a soft silicone lip.

In another aspect, the invention features a microdermabrasion system, including a microdermabrasion apparatus having a housing having a first end and a second end, a cylindrical wall located generally adjacent the first end, a motor located within the housing, the motor including a shaft, a pump located within the housing and a tube located within the housing and connected between the pump and the cylindrical wall, a power entry located on the second end and coupled to the motor and the pump, a switching mechanism coupled to the power entry, the pump and the motor, a suction cup connected to the wall and an exfoliation tip connected to the motor shaft.

One advantage of the invention is that it is compact and easy to use.

Another advantage of the invention is that is removed dead impacted skin cells and other debris.

Another advantage of the invention is that it provides increased blood flow to the treated dermis.

Another advantage of the invention is that it facilitates the benefits of lotions applied to the skin.

Another advantage of the invention is that it stimulates the growth of new skin cells.

Another advantage is that the vacuum pump and disk motor are decoupled.

Another advantage is that the rotating abrading disk stops rotating when it is not in motion over the user's skin.

Other objects, advantages and capabilities of the invention will become apparent from the following description taken in conjunction with the accompanying drawings showing the preferred embodiment of the invention.

### BRIEF DESCRIPTION OF THE DRAWINGS

FIG. 1A illustrates a perspective view of an embodiment of a microdermabrasion apparatus;

FIG. 1B illustrates a perspective view of constituent components of an embodiment of a microdermabrasion apparatus;

FIG. 2 illustrates an internal side view of an embodiment of a microdermabrasion apparatus;

FIG. 3 illustrates a side view of internal constituent components of an embodiment of a microdermabrasion apparatus;

FIG. 4 illustrates a close up side view of an embodiment of a microdermabrasion apparatus tip;

FIG. 5 illustrates a close up side view of an embodiment of another microdermabrasion apparatus tip;

FIG. 6 illustrates a perspective view of an embodiment of a microdermabrasion apparatus tip;

FIG. 7 illustrates a perspective view of another embodiment of a microdermabrasion apparatus tip; and

FIG. 8 illustrates a perspective view of another embodiment of a microdermabrasion apparatus tip.

### DETAILED DESCRIPTION

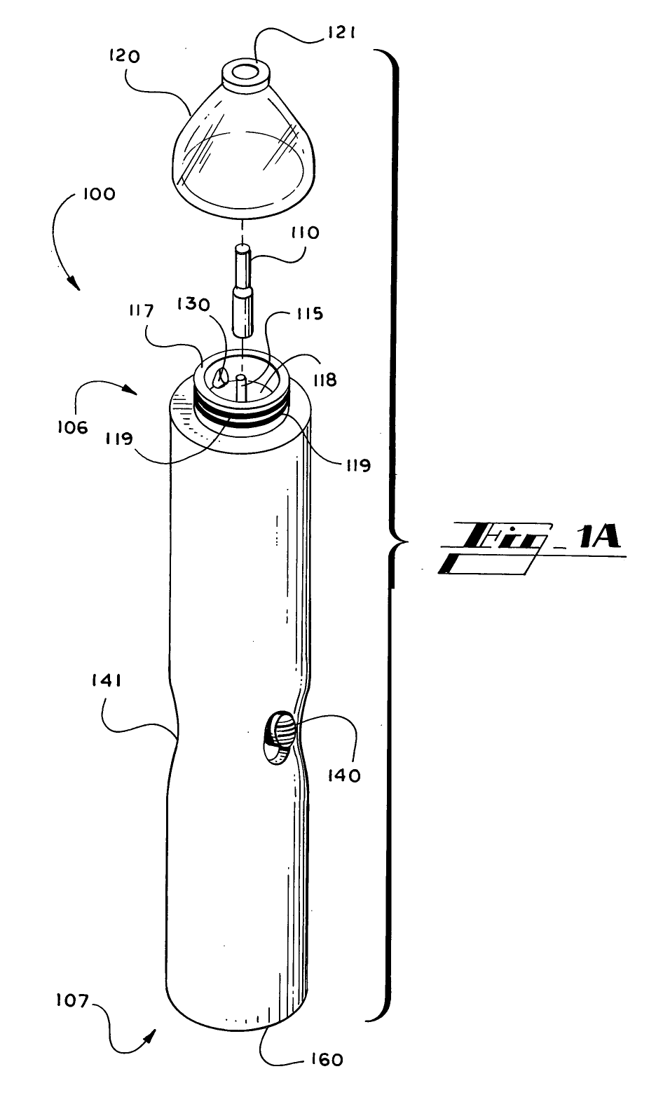

Referring to the drawings wherein like reference numerals designate corresponding parts throughout the several figures, reference is made first to FIG. 1A that illustrates a perspective view of an embodiment of a microdermabrasion apparatus 100 having a first end 106 and second end 107. The first end 106 typically includes a suction/vacuum cup 120 having a ridge 121 that is adapted to come into contact and form a area of suction with a user's skin. The second end 107 of the housing 105 is typically equipped with a power entry 160 that is shown in further detail below. The power entry 160 can be located at other areas of the housing 105. In one embodiment, the power entry 160 is a 12 volt DC port to receive a 12 volt DC power source. In other embodiments, the power entry 160 can receive other forms of power. In still another embodiment, the apparatus 100 can be powered by a battery located within the housing. In one implementation, the battery can be rechargeable.

The apparatus further includes an exfoliation tip 110, which is typically an abrasive disc made from a suitable abrasive material. The exfoliation tip 110 can have a variety of sizes and shapes. Several embodiments of the exfoliations tip 110 are described in the discussion below. The exfoliation tip 110 is typically located within and surrounded by the suction cup 120. The exfoliation tip 110 is mechanically coupled to a shaft 115, the shaft 115 being mechanically coupled to a motor 125, that is internal to the housing 105. The exfoliation tip 110 spins about a central axis of the shaft 115. In general, the first end 106 of the housing further includes a generally cylindrical wall 117. The wall 117 generally surrounds the shaft 115 that protrudes from the housing 105. The wall generally surrounds a space 118 that generally defines the area in which a vacuum can be formed. The wall 117 can further include one or more seals 119 generally oriented parallel to each other and around the perimeter of the wall 117. In a typical embodiment, the seals 119 are o-rings that can be located within perimeter trenches adapted to receive the o-rings. The wall 117 is further described in the discussion below. In general, the suction cup 120 can be placed over the wall 117 and the seals 119. The connection between the suction cup 120 and the seals 119 is such that a good seal is created between the wall 117 and the suction cup 120 when the suction cup 120 is connected.

The apparatus 100 further includes a tubing 130 located within the housing 105 and protruding into the space 118 enclosed by the wall 117. The tubing 130 is generally connected between the suction cup 120 having a contact ridge 121 and a pump 150 that is also located within the housing 105 and described in further detail in the discussion below.

The apparatus 100 can further include a power switch 140 that can be located at any location on the housing 105. In a typical embodiment, the switch 140 is located at a generally central location on the housing that provides an ergonomic location from which the user can engage the switch with his thumb or other finger. The switch 140 can be generally located within a depression 141 located generally in the middle and along the perimeter of the housing 105.

FIG. 1B illustrates a perspective view of constituent components of an embodiment of a microdermabrasion apparatus 100. The housing 105 can be generally defined by two halves 108, 109. The halves 108, 109 can be connected together to form the housing 105 that encloses the internal components of the apparatus 100. The internal components include the motor 125 as mentioned in the above description. The motor 125 typically provides rotational force to the exfoliation tip 110 so as to rotate the exfoliation tip 110 within the suction cup 120. The motor 125 can also provide other types of force to the shaft 115 and the exfoliation tip 110 such as a vibrational force. The motor 125 is generally coupled to the power entry 160 and to the power switch 140 that is located within the housing 105 and exposed to the exterior of the housing 105 so that a user can manipulate the switch 140. The switch 140 can be engaged so as to provide power to the motor 125 to energize the exfoliation tip 110 as well as to provide power to the vacuum pump 150. The switch 140 is coupled to an internal switching mechanism 145 that can be mechanically displaced to complete an electrical connection among the switching mechanism 145, the motor 125 and the pump 150, thereby providing electrical power to the same. In general, the power entry 160 is coupled to the motor 125 and the pump 150 as described in further detail below.

The tube 130 is connected between the wall 117 and the pump 150. The pump 150 provides a vacuum pull through the tube 120 and onto the space 118 in the wall 117. As discussed in further detail in the description below, the suction cup 120 is connected around the wall 117 and the seals 119. In this view, the seals 119 have been removed from illustrative purposes, illustrating the trenches 101. The suction cup 120 effectively increases the size of the space 118 thereby providing a greater volume through which the vacuum can be pulled. The pump 150 is also electrically coupled to the power entry 160 and to the switching mechanism 145. The pump 150 can be activated when the user engages the switch 140. The tube 130 holds the vacuum pull between the pump 150 and the suction cup 120. Therefore, for reasons described further below, the tube 130 further includes a filter 135 located within the tube 130 and between the pump 150 and the suction cup 120. In addition, the tube 130 typically includes a vent 136 as discussed in further detail in the description below.

FIG. 1B further illustrates that the halves 108, 109 can include a series of cradles to hold the various internal components in place. A motor cradle 126 holds the motor 125 in place when the halves 108, 109 are connected. A switching mechanism cradle 146 holds the switching mechanism 145 in place when the halves 108, 109 are connected. A pump cradle 151 holds the pump 150 in place when the halves 108, 109 are connected. It is understood that various additional cradles are contemplated to hold various other components in place when the halves 108, 109 are connected to form the housing 105. The halves 108, 109 further include a series of feed-through connectors 111 that allow connecting devices such as rivets and screws to be used to connect the halves 108, 109 to one another.

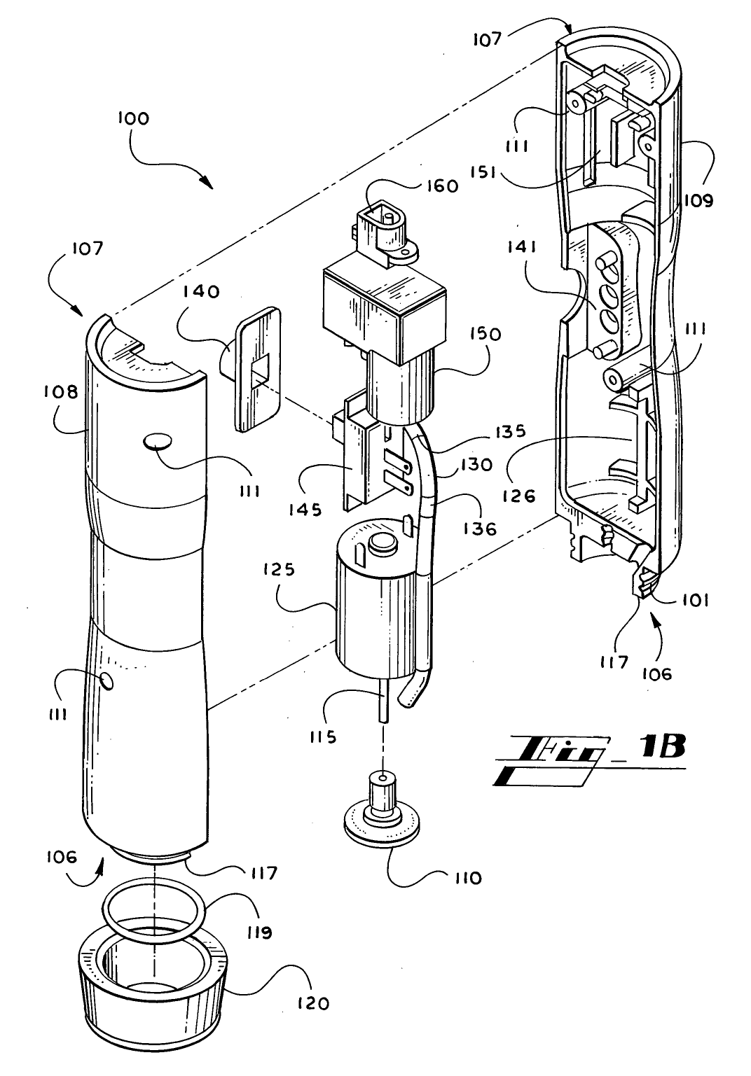

FIG. 2 illustrates an internal side view of an embodiment of a microdermabrasion apparatus 100. As described above, the internal components include the motor 125, the pump 150, the switching mechanism 145, the tube 130 and the power entry 160. The additional components of the apparatus 100 include the housing 105, the suction cup 120, the exfoliation tip 110. As is discussed in further detail below, the exfoliation tip 110 is offset inside the space 118 by a distance D. The distance D is measured from the end of the exfoliation tip 110 to a plane P generally defined at the end of the suction cup 120. Furthermore, an opening 122 is generally defined by the ridge 121.

In a typical embodiment, the motor 125 is located generally adjacent the first end 106 so that the exfoliation tip 110 can be easily coupled to the shaft 115 within the space 118. The pump 150 is typically located adjacent the second end 107 and adjacent the power entry 160. The switching mechanism 145 is typically located toward the middle of the apparatus 100 generally located adjacent the depression 141. As described above, one end of the tube 130 opens into the space 118 adjacent the shaft 115, at the first end 106. The tube 130 is coupled to the pump 150. Since the pump 150 is located adjacent the second end 107, the tube 130 is positioned along a substantial length of the housing 105. In a typical implementation, in order to provide a smooth path and prevent kinks and bends from forming in the tube 130, the tube 130 can be directed generally from a pump input 151 to the space 118 taking various curves along its path past the switching mechanism 145 and motor 125.

FIG. 3 illustrates a side view of internal constituent components of an embodiment of a microdermabrasion apparatus 100. In various descriptions above, it has been stated that the motor 125, the pump 150, the power entry 160 and the switching mechanism 145 are electrically coupled. In this view, the constituent components are laid out as described above. In addition, for illustrative purposes various other features and components of the apparatus have been removed to show the various terminals, leads and connections. The switching mechanism 145 includes terminals 147. The motor 125 includes terminals 127. The pump 150 includes terminals 152. In general, leads 180 connect the various terminals. For example, the terminals 127 are coupled to the terminals 147, 152, 161. As described above, the electrical coupling allows power to be delivered from the power input 160 to both the motor 125 and pump 150 and generally regulated by the switching mechanism 145. It is understood that many lead configurations are contemplated in other embodiments.

The apparatus 100 can be implemented by a user in a variety of ways. In one implementation, the user can place the ridge 121, which can be a soft silicone lip, of the suction cup 120 in contact with the user's skin. The user then can engage with switch 140 thereby providing power to the motor 125 and the pump 150. As the pump 150 pulls a vacuum through the tube 130 on the space 118 within the wall 117 and the suction cup 120, the user's skin is pulled partially into the suction cup 120 where a vacuum is formed within the space 118 in the suction cup 120. At the same time, the exfoliation tip 110 is provided with power, typically a rotation force. As the user's skin is pulled within the suction cup 120, the skin also comes into contact with the exfoliation tip 110 thereby exfoliating the user's skin. As dead skin cells and other debris are removed by the exfoliation tip 110, the debris gathers inside the suction cup 120. The vacuum pulled on the suction cup 120 serves also to pull the debris into the tube 130. To prevent the pump 150 from becoming soiled with this debris, the filter 135 acts to pull the debris from the tube, thereby preventing it from entering the pump 150. In a typical implementation, the filter can be a 5 micron filter to filter the smallest pieces of debris. It is understood that in other implementation, the filter 135 can have other specifications. In another embodiment, the motor can be equipped with a shut down switch if too much pressure is applied by the skin on the exfoliation tip 110.

In a typical embodiment, although the motor 125 and the pump 150 receive power from the same source, the motor 125 and the pump 150 are de-coupled from one another and run independently from one another. In a typical implementation, the output of the motor 125 and the size of the tip 110 are directly related as in a torque valve. Therefore, if the user's skin comes into full contact with the tip 110, while the entire apparatus 100 is not being moved along the skin's surface, the tip 110 typically ceases rotation. When the user begins to move the apparatus 100 along the skin again, the tip 110 then begins to rotate once again. Furthermore, in a typical embodiment, the pump 150 is a piston style vacuum pump that is an tunable through a venting process utilizing the vent 136. In general, the vent 136 is a hole that can be pre-tuned, generally by varying the size, in order to provide a desirable vacuum.

In general, the exfoliation tip 110 is useful for removing dead impacted skin cells which in turn helps to promote the growth of new skin cells. The suctioning provided by the apparatus helps to promote increased blood flow into the treated dermis. The removal of the dead skin cells generally facilitates the benefit of lotions applied to the skin.

Referring again to FIG. 2, the exfoliation tip 110 typically includes a main body 110a that connects to the shaft 115 and a abrading disk 110b connected to the main body. The abrading impregnated surface disk 110b includes an abrasive media. In a typical embodiment, the abrasive media can include but is not limited to diamond, sapphire or any other gem, aluminum oxide, cobalt, carbide, tungsten and the like.

In typical operation, the user's skin must be pulled into contact with the exfoliation tip 110. In general, the plane P defines the plane where the surface of the user's skin is located prior to powering the apparatus 100. As such, it is appreciated that the surface of the skin is separated from the exfoliation tip 100 by the distance D. Therefore, by putting the skin in contact with the ridge 121, a seal is formed between the surface of the skin and the ridge. When the apparatus 100 is powered, the pump 150 pulls a vacuum through the tube 130 and into the space 118 defined within the wall 117 and the suction cup 120. When the vacuum is pulled, the user's skin penetrates the plane P and is pulled partially into the space 118 through the distance D and in contact with the exfoliation tip 110. The seal formed between the skin and the ridge 121 keeps the vacuum in the space 118.

As described above, in a typical implementation, the output of the motor 125 and the size of the tip 110 are directly related as in a torque valve. Therefore, if the user's skin comes into full contact with the tip 110, while the entire apparatus 100 is not being moved along the skin's surface, the tip 110 typically ceases rotation. When the user begins to move the apparatus 100 along the skin again, the tip 110 then begins to rotate once again. During use, the dead skins cells and other debris gather in the space 118 as the user moves the apparatus 100 along the surface of the skin. In a typical implementation, the suction cup 120 can be removed and washed as needed. In addition, the wall 117 can also be rinsed to remove debris. As described above, the filter 135 prevents debris from entering and damaging the pump 150. Furthermore, after each use, the exfoliation tip is generally adapted to be disposable and for single use only. As such, the apparatus 100 can include many additional exfoliation tips 110 as a kit.

The apparatus can also include other skin care attachments. For example, the apparatus 100 can include a pore cleansing tip, massager tips, buffer tips for finger and toe nails, sander tips for finger and toe nails, a pumice stone attachment for treatment of calluses, an abrasive cup design, a vibrational tip, an ultra small vacuum cup for pore cleansing and the like. Any of the attachments can be included with the general apparatus as a kit. Any further combinations of the attachments can also be combined as a kit with the apparatus or as an accessory pack.

In a typical embodiment, a microdermabrasion kit can include several different suction cups 120 of varying sizes and various exfoliation disks 110 of varying sizes. In general, small exfoliation tips 110 can be used around the eyes, nose and mouth and large tips can be used for larger parts of the body, bust area legs and the like. As mentioned above, the exfoliation tips 110 are typically for single use only. Therefore, several exfoliation tips of varying sizes can be included in the kit.

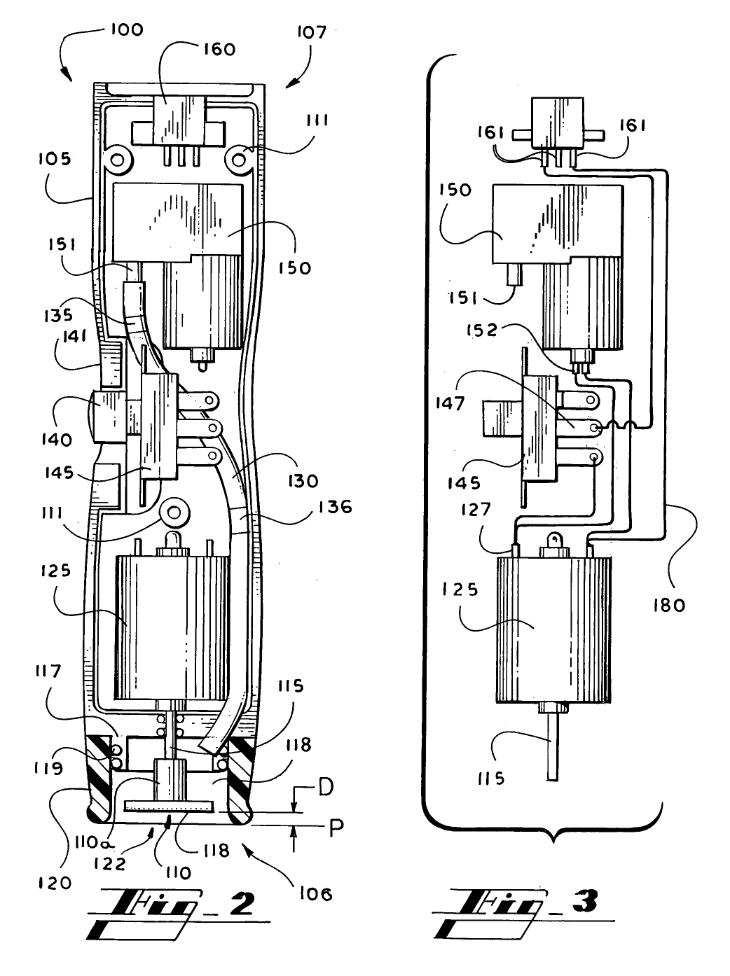

FIG. 4 illustrates a close up side view of an embodiment of a microdermabrasion apparatus tip 110. A portion of the housing 105, the wall 117, the seals 119, the shaft and the tube 130 are also shown. The exfoliation tip 110 shown in FIG. 2 above is a large tip as compared to the small tip illustrated in FIG. 4. As such, the suction cup 120 is proportionally smaller with a narrower opening 122. As described above, the exfoliation tip 110 includes the main body 110a and the abrading impregnated surface disk 110b. Typically, the smaller exfoliation tip 110 and suction cup 120 can be used in smaller and “harder to access” areas of the body.

FIG. 5 illustrates a close up side view of an embodiment of another microdermabrasion apparatus tip 110. A portion of the housing 105, the wall 117, the seals 119, the shaft and the tube 130 are also shown. The exfoliation tip 110 shown in FIG. 2 above is a large tip as compared to the medium tip illustrated in FIG. 5. As such, the suction cup 120 is proportionally smaller with a narrower opening 122. As described above, the exfoliation tip 110 includes the main body 110a and the abrading impregnated surface disk 110b. Typically, the medium exfoliation tip 110 and suction cup 120 can be used in smaller and “harder to access” areas of the body, but larger than the smaller tip shown in FIG. 4.

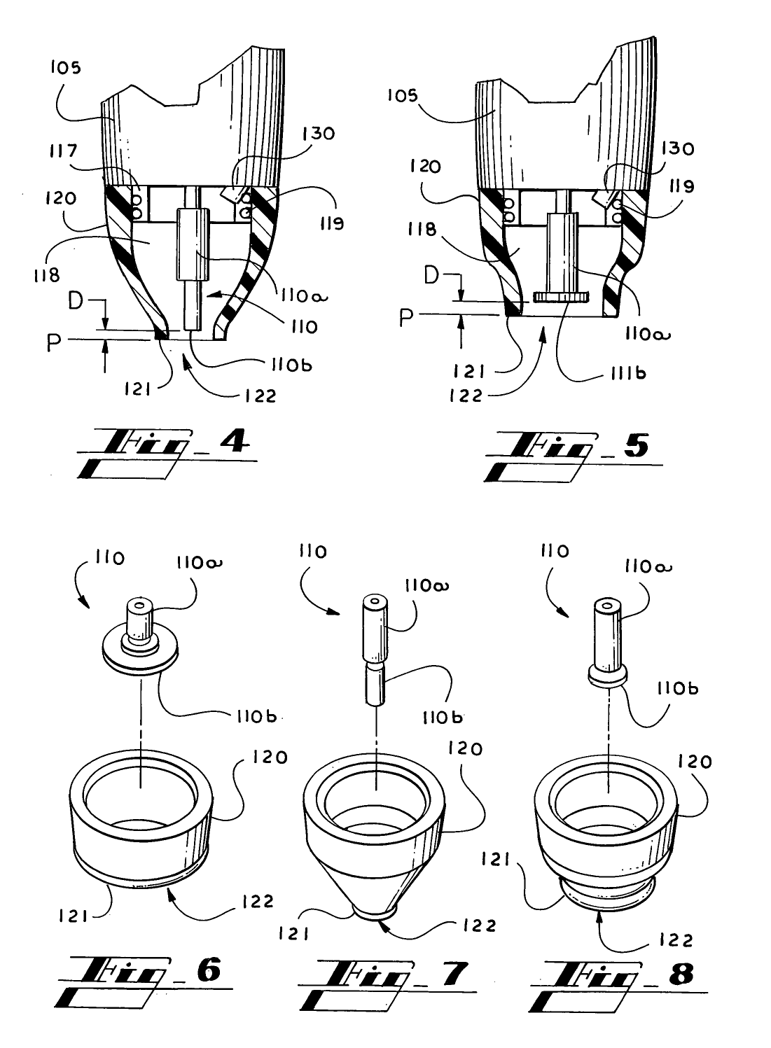

FIG. 6 illustrates a perspective view of an embodiment of a large exfoliation tip 110 having a main body 110a and an abrading impregnated surface disk 110b, and a suction cup 120 having a ridge 121 and an opening 122.

FIG. 7 illustrates a perspective view of another embodiment of a small exfoliation tip 110 having a main body 110a and an abrading impregnated surface disk 110b, and a suction cup 120 having a ridge 121 and an opening 122.

FIG. 8 illustrates a perspective view of another embodiment of a medium exfoliation tip 110 having a main body 110a and an abrading impregnated surface disk 110b, and a suction cup 120 having a ridge 121 and an opening 122.

In general, the exfoliation tips 110 and respective suction cups 120 described above are proportional and allow focused exfoliation of desired areas. The openings 122 of the respective suction cups 120 decrease with the decreasing size of the exfoliation tips 110 or increase with the increasing size of the exfoliation tips 110 in order to allow the user to have the focused exfoliation. Although the exfoliation tips 110 and the suction cups 120 can be mixed and matched, it is generally desirable to use the respective suction cup 110 with the appropriate exfoliation tip 110. For example, although the suction cup 120 with the largest opening 122 can be used with the smallest exfoliation tip 110, an unnecessarily large amount of surface area of the skin can be pulled into the space 118 during use making it difficult to focus the exfoliation tip on the desired area. By using the suction cup 120 with the smaller opening 122 in this example, the user can properly place the ridge 121 in the desired area that corresponds accurately with the desired area to exfoliate suing the smaller exfoliation tip 110.

It is understood that the foregoing is considered as illustrative only of the principles of the invention. Further, various modifications may be made of the invention without departing from the scope thereof and it is desired, therefore, that only such limitations shall be placed thereon as are imposed by the prior art and which are set forth in the appended claims.

What is claimed is:

1. A microdermabrasion apparatus, comprising:
a housing having a first end and a second end;
a suction cup located at the first end of the housing;
a motor located within the housing;
a pump located within the housing;
an exfoliation tip mechanically coupled to a shaft that is mechanically coupled to the motor, the exfoliation tip being located within a vacuum space in the suction cup; and
a tube located within the housing and connected between the pump and the vacuum space.

2. The apparatus as claimed in claim 1 further comprising a power entry optionally connected to the second end of the housing, the power entry being electrically coupled to a switch located on the housing, to the pump and to the motor.

3. The apparatus as claimed in claim 1 further comprising a cylindrical wall located adjacent the first end and within a portion of the suction cup.

4. The apparatus as claimed in claim 3 further comprising a seal located around the perimeter of the wall and in between the wall and a portion of the suction cup.

5. The apparatus as claimed in claim 4 wherein the seal is an o-ring.

6. The apparatus as claimed in claim 3 wherein the tube protrudes from a portion of the wall into the vacuum space.

7. The apparatus as claimed in claim 1 further comprising a filter located within the tube.

8. The apparatus as claimed in claim 7 further comprising a vent located in the tube between the filter and the vacuum space.

9. The apparatus as claimed in claim 1 wherein the exfoliation tip comprises a main body connected to the shaft and an abrading impregnated surface disk connected generally perpendicular to the main body.

10. The apparatus as claimed in claim 9 wherein the abrading impregnated surface disk further comprises an abrasive media.

11. The apparatus as claimed in claim 1 wherein the suction cup further comprises an opening generally defined by a ridge.

12. The apparatus as claimed in claim 11 wherein the exfoliation tip is offset inside the vacuum space and from the ridge by a distance.

13. A microdermabrasion kit, comprising:
a microdermabrasion apparatus comprising:
a housing having a first end and a second end;
a cylindrical wall located generally adjacent the first end;
a motor located within the housing, the motor including a shaft oriented about a central axis;
a pump located within the housing; and
a tube located within the housing and connected between the pump and the cylindrical wall;
a suction cup adapted to be connected to the wall; and
an exfoliation tip adapted to be connected to the motor shaft, and further adapted to spin about the central axis of the shaft.

14. The kit as claimed in claim 13 wherein the exfoliation tip is adapted to be located within a vacuum space formed when the suction cup is placed over the wall and the pump pulls a vacuum on the space, the exfoliation tip being further adapted to spin when the motor is supplied power.

15. The kit as claimed in claim 14 wherein the exfoliation tip comprises an abrading impregnated surface disk.

16. The kit as claimed in claim 13 further comprising a pore cleansing tip adapted to be connected to the shaft.

17. The kit as claimed in claim 13 further comprising a buffer tip for finger nails and toe nails adapted to be connected to the shaft.

18. The kit as claimed in claim 13 further comprising a sander tip for finger nails and toe nails adapted to be connected to the shaft.

19. The kit as claimed in claim 3 further comprising a pumice stone adapted to be connected to the shaft.

20. The kit as claimed in claim 13 further comprising a vibrational tip adapted to be connected to the shaft.

21. The kit as claimed in claim 13 wherein the suction cup is abrasive.

22. The kit as claimed in claim 13 wherein the suction cup includes an opening generally defined by a ridge.

23. The kit as claimed in claim 22 wherein the ridge includes a soft silicone lip.

24. A microdermabrasion system, comprising:
a microdermabrasion apparatus comprising:
a housing having a first end and a second end;
a cylindrical wall located generally adjacent the first end;
a motor located within the housing, the motor including a shaft;
a pump located within the housing; and
a tube located within the housing and connected between the pump and the cylindrical wall;
a power entry located on the second end and coupled to the motor and the pump;
a switching mechanism coupled to the power entry, the pump and the motor;
a suction cup connected to the wall; and
an exfoliation tip connected to the motor shaft.
